(19) United States
(12) Patent Application Publication
(10) Pub. No.: US 2010/0114332 A1
(43) Pub. Date: May 6, 2010

(54) ANKLE JOINT STRUCTURE OF WALKING ASSISTANCE DEVICE

(75) Inventors: Jun Ashihara, Wako-shi (JP); Tatsuya Noda, Wako-shi (JP); Hiroshi Kudoh, Wako-shi (JP); Yasushi Ikeuchi, Wako-shi (JP); Yutaka Hiki, Wako-shi (JP)

Correspondence Address: RANKIN, HILL & CLARK LLP, 38210 Glenn Avenue, WILLOUGHBY, OH, 44094-7808, US

(73) Assignee: HONDA MOTOR CO., LTD., Tokyo (JP)

(21) Appl. No.: 12/610,699

(22) Filed: Nov. 2, 2009

(30) Foreign Application Priority Data
Nov. 6, 2008 (JP) 2008-285547

(51) Int. Cl. A61F 2/66

(52) U.S. Cl. 623/47

(57) ABSTRACT

An ankle joint structure of a walking assistance device is capable of protecting a user from feeling uncomfortable by properly following the foot motions of the user. An ankle joint has a turn shaft member enabling a ground contact assembly to turn relative to a leg link, a lateral rotation shaft member enabling the ground contact assembly to laterally rotate relative to the leg link, and a longitudinal rotation shaft member enabling the ground contact assembly to longitudinally rotate relative to the leg link. The turn shaft member is connected to the leg link, the longitudinal rotation shaft member is connected to the ground contact assembly, and the lateral rotation shaft member is disposed between the turn shaft member and the longitudinal rotation shaft member.

### BACKGROUND OF THE INVENTION

1. Field of the Invention

The present invention relates to an ankle joint structure of a walking assistance device which reduces load acting on a leg or legs of a user, thereby assisting the user in walking.

2. Description of the Related Art

Hitherto, there has been known a walking assistance device which has a load transmit assembly, ground contact assemblies, and leg links provided between the load transmit assembly and the ground contact assemblies, and which supports at least a part of the weight of a user by the leg links through the intermediary of the load transmit assembly (refer to, for example, Japanese Patent Application Laid-Open No. 2008-73506, which will be hereinafter referred to as patent document 1).

Each of the ground contact assemblies and each of the leg links of the walking assistance device disclosed in patent document 1 are connected by an ankle joint. The ankle joint includes a lateral rotation shaft member, which enables toe ground contact assembly to rotate in a lateral direction relative to the leg link, a turn shaft member, which enables the ground contact assembly to turn relative to the leg link, and a longitudinal rotation shaft member, which enables the ground contact assembly to rotate in a longitudinal direction relative to the leg link, these three shafts being disposed in this order beginning at the top. Thus, the ankle joint is constructed to have three degrees of freedom.

There has been known another ankle joint constructed to have three degrees of freedom by connecting a ground contact assembly and a leg link by a spherical joint (refer to, for example, paragraph [0048] and FIG. 3 in Japanese Patent Application Laid-Open No. 2007-54616, which will be hereinafter referred to as patent document 2).

In the ankle joint disclosed in the aforesaid patent document 1, as a user turns his/her foot, the longitudinal rotation axial line of the longitudinal rotation shaft member and the lateral rotation axial line of the lateral rotation shaft member gradually approach a parallel state. Hence, in the state wherein the user has turned his/her foot, if, for example, the user attempts to rotate the foot in the longitudinal direction, then a slight discrepancy may develop between a motion of the user's foot and a motion of the ground contact assembly, causing the user to feel uncomfortable.

According to the ankle joint disclosed in the aforesaid patent document 2, limited operating angles due to the construction of the spherical joint may lead to failure to properly respond to foot motions of a user, making the user feel discomfort.

### SUMMARY OF THE INVENTION

In view of the problems described above, an object of the present invention is to provide an ankle joint structure of a walking assistance device capable of properly following foot motions of a user, thereby avoid making the user feel uncomfortable.

To this end, a first aspect of the present invention provides an ankle joint structure used with a walking assistance device, which includes a load transmit assembly, a ground contact assembly, and a leg link provided between the load transmit assembly and the ground contact assembly to support at least a part of the weight of a user by the leg link through the intermediary of the load transmit assembly, the ankle joint structure connecting the ground contact assembly and the leg link. The ankle joint structure comprises a turn shaft member, which enables the ground contact assembly to turn relative to the leg link, a longitudinal rotation shaft member, which enables the ground contact assembly to rotate in a longitudinal direction relative to the leg link, and a lateral rotation shaft member, which enables the ground contact assembly to rotate in a lateral direction relative to the leg link, wherein the turn shaft member is disposed above or below the longitudinal rotation shaft member and the lateral rotation shaft member.

According to the first aspect of the present invention, the turn shaft member is disposed above or below the longitudinal rotation shaft member and the lateral rotation shaft member, so that the longitudinal rotation shaft member and the lateral rotation shaft member are connected without the intermediary of the turn shaft member. Thus, unlike the conventional ankle joint structures, when the ground contact assembly turns relative to the leg link at the turn shaft member, the longitudinal rotation axial line of the longitudinal rotation shaft member and the lateral rotation axial line of the lateral rotation shaft member do not approach the parallel state. This enables the ankle joint to properly follow foot motions of a user, making it possible to protect the user from feeling uncomfortable.

A second aspect of the present invention provides an ankle joint structure used with a walking assistance device, which includes a load transmit assembly, a ground contact assembly, and a leg link provided between the load transmit assembly and the ground contact assembly to support at least a part of the weight of a user by the leg link through the intermediary of the load transmit assembly, the ankle joint structure connecting the ground contact assembly and the leg link through the intermediary of a spherical joint. The ankle joint structure includes a turn shaft member, which enables the ground contact assembly to turn relative to the leg link, wherein one end of the turn shaft member is connected to one of the ground contact assembly and the leg link, while the other end of the turn shaft member swingably retains a sphere of the spherical joint, and a joint shaft member provided on the sphere is connected to the other of the ground contact assembly and the leg link.

According to the second aspect of the present invention, the provision of the turn shaft member permits a larger operating angle in a turning direction with a larger rotational angle than that of the longitudinal rotation shaft member and the lateral rotation shaft member, and the spherical joint ensures a rotation with a smaller rotational angle in the longitudinal direction and the lateral direction than that of the turn shaft member. This allows the motion of the ground contact assembly relative to a leg link to properly follow a foot motion of the user.

In both first and second aspects of the present invent on, the shaft which rotates an ankle to allow a foot of a user to turn outward and inward in an upright posture state in which the walking assistance device is standing with both ground contact assemblies in contact with a ground surface of a floor surface or the like is defined as a turn shaft, a shaft which rotates an ankle to move up or down a tiptoe of a foot of the user in the upright posture state is defined as the longitudinal rotation shaft, and a shaft which rotates a foot of the user in the upright posture state in the lateral direction, taking the ankle as a support point, is defined as the lateral rotation shaft.

### BRIEF DESCRIPTION OF THE DRAWINGS

FIG. 1 is a side view illustrating a first embodiment of an ankle joint structure of a walking assistance device in accordance with the present invention;

FIG. 2 is a front view of the walking assistance device of the first embodiment;

FIG. 3 is a sectional view of the ankle joint structure of the walking assistance device of the first embodiment taken at line in FIG. 1;

FIG. 4 is a sectional view of the ankle joint structure of the walking assistance device of the first embodiment taken at line IV-IV in FIG. 2;

FIG. 5 is a side view illustrating a second embodiment of the ankle joint structure of the walking assistance device in accordance with the present invention;

FIG. 6 is a front view of the walking assistance device of the second embodiment; and

FIG. 7 is a sectional view of the ankle joint structure of the walking assistance device of the second embodiment taken at line VII-VII in FIG. 5.

### DESCRIPTION OF THE PREFERRED EMBODIMENTS

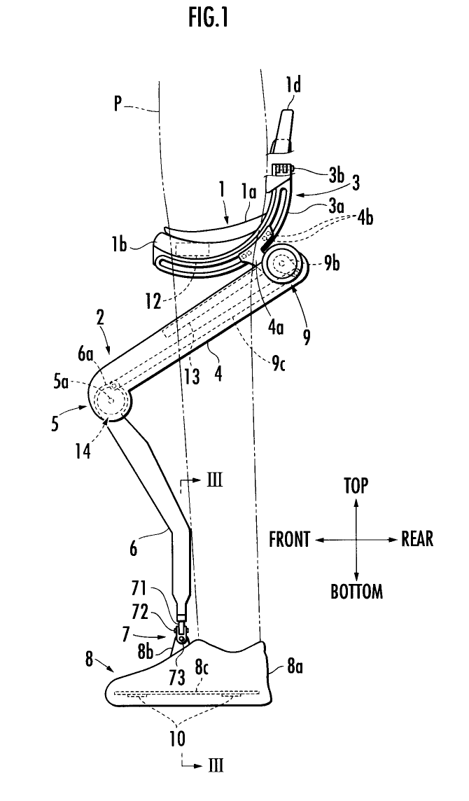

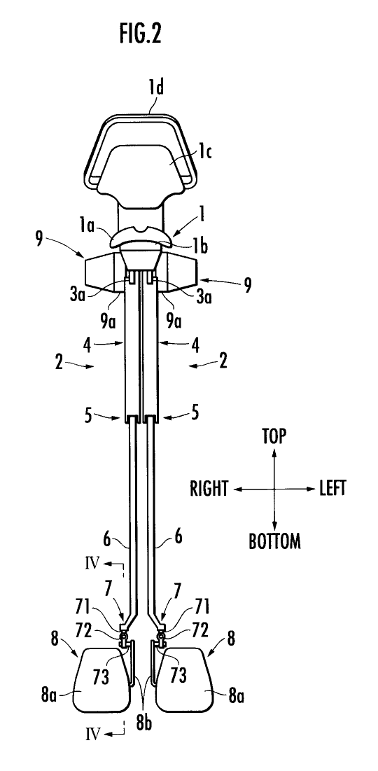

The following will describe a walking assistance device of a first embodiment to which an ankle joint structure in accordance with the present invention has been applied, with reference to FIG. 1 to FIG. 3. As illustrated in FIG. 1 and FIG. 2, the walking assistance device according to the first embodiment has a seating member 1 serving as a load transmit assembly on which a user P sits astride and a pair of right and left leg links 2 and 2 connected to the seating member 1.

Each of the leg links 2 is constituted of a bendable link having a first link member 4, which is connected to a hip joint 3 provided on the seating member 1, and a second link member 6, which is connected to a lower end of the first link member 4 through a rotary knee joint 5. Further, a ground contact member 8 (ground contact assembly) to be attached to each of right and left feet of the user P is connected to a lower end of the second link member 6 through ac ankle joint 7.

Each of the leg links 2 is further provided with a drive source 9 for the knee joint 5. The knee joint 5 is rotationally driven by the drive source 9 to drive each of the leg links 2 in a stretching direction, that is, in the direction for pushing the seating member 1 up, thereby generating a force for pushing the seating member 1 up (hereinafter referred to as “the load support force”). The Load support force generated in the leg link 2 is transferred to the body trunk of the user P through the intermediary of the seating assembly 1. More specifically, a part of the weight of the user P is supported by the leg links 2 and 2 through the intermediary of the seating member 1 by the load support force, thus reducing the load acting on the legs of the user P.

The seating member 1 is composed of a saddle-shaped seat 1a, on which the user P sits, a support frame 1b on a lower surface supporting the seat 1a, and a hip pad 1c attached to a rising portion at the rear end of the support frame 1b, which rises at the rear of the seat 1a. The hip pad 1c is provided with an arcuate handle 1d which can be grasped by the user P.

Further, the seating member 1 has an arcuate guide rail 3a constituting the hip joint 3 for the leg links 2. The leg links 2 are movably engaged with the guide rail 3a through the intermediary of a plurality of rollers 4b rotatably attached to a slider 4a fixed on the upper end of the first link member 4. Thus, each of the leg links 2 swings in the longitudinal direction about the curvature center of the guide rail 3a. The supporting point of the swing of each of the leg links 2 in the longitudinal direction provides the curvature center of the guide rail 3a.

Further, the guide rail 3a is rotatably supported by a rising portion of the rear end of the support frame 1b of the seating member 1 through the intermediary of a longitudinal support shaft 3b. Hence, the guide rail 3a is connected to the seating member 1 such that the guide rail 3a may swing in the lateral direction. This allows each of the leg links 2 to swing in the lateral direction, enabling a leg of the user P to be abducted. The curvature center of the guide rail 3a and the axial line of the support shaft 3b are positioned above the seat 1a. This makes it possible to prevent the seating member 1 from significantly tilting vertically or laterally when the weight of the user P shifts.

The drive source 9 is constituted of an electric motor with a speed reducer 9a mounted on the outer surface of an upper end portion of the first link member 4 of each of the leg links 2. A drive crank arm 9b on an output shaft of the speed reducer 9a is connected with a driven crank arm 6a secured to the second link member 6 coaxially with a joint shaft 5a of the knee joint 5 through the intermediary of a connection link 9c. With this arrangement, the motive power output from the drive source 9 through the intermediary of the speed reducer 9a is transferred to the second link member 6 through the intermediary of the connection link 9c. Then, the second link member 6 swings about the joint shaft 5a relative to the first link member 4, causing the leg link 2 to bend or stretch.

Each of the ground contact members 8 has a shoe 8a and a connection member 8b, which is secured to the shoe 8a and which extends upward. Further, the second link member 6 of the leg link 2 is connected to the connection member 8b through the intermediary of a three-axis third joint 7. As illustrated in FIG. 1, a pair of front and rear pressure sensors 10 and 10 for detecting load acting on a metatarsophalangeal joint (MP joint) portion and a heel portion of a foot of the user P is installed on the bottom surface of an insole 8c provided in the shoe 8a. In addition, a two-axis force sensor (not shown) is built in the ankle joint 7. The support frame 1b of the seating member 1 incorporates a controller 12, which is a control means, while the first link member 4 incorporates a battery 13.

The detection signals of the pressure sensors 10 and the force sensor are input to the controller 12. Then, the controller 12 controls the drive source 9 on the basis of the signals from these pressure sensors 10 and the force sensor to drive the knee joint 5, thereby executing the walking assistance control for generating the aforesaid load support force.

Here, the load support force acts on a line which connects the support point of the longitudinal swing of the leg link 2 at the hip joint 3 and the support point of the longitudinal swing of the leg link 2 at the ankle joint 7, as observed from a lateral direction (hereinafter referred 2C to as “the reference line”). Therefore, in the walking assistance control, an actual load support force acting on the reference line (to be precise, the resultant force of a load support force and the force from the weights of the seating member 1 and the leg links 2) is calculated on the basis of the detection values of the forces in the directions of two axes detected by the force sensor. Further, based on the detected pressures of the pressure sensors 1) of each of the ground contact members 8, the ratio of the acting load of each foot in relation to the total load acting on both feet of the user P is calculated. Subsequently, a value obtained by multiplying a set value of a load support force, which is set beforehand, by the ratio of the load of each foot is calculated as a control target value of the load support force to be generated at each of tee leg links 2. Then, the drive source 9 is controlled such that the actual load support force calculated on the basis of the detection values of the force sensor agrees with the control target value.

In the case where the ankle joint 7 is constituted of a turn shaft enabling the ground contact member 8 to turn relative to the leg link 2, a longitudinal rotation shaft member, which is rotatable in the longitudinal direction, and a lateral rotation shaft member, which is rotational in the lateral direction, wherein the shaft which rotates an ankle to allow a foot of a user to turn outward and inward in an upright posture state in which the walking assistance device is standing with both ground contact assemblies in contact with a ground surface or a floor surface or the like is defined as the turn shaft, the shaft whish rotates an ankle to move up or down a tiptoe of a foot of the user in the upright posture state is defined as the longitudinal rotation shaft, and the shaft which turns a foot of the user in the upright posture state in the lateral direction, using the ankle as a support point, is defined as the lateral rotation shaft, and if the lateral rotation shaft member, the turn shaft member, and the longitudinal rotation shaft member are disposed in this order beginning at the top, then the lateral rotation axial line of the lateral rotation shaft member and the longitudinal rotation axial line of the longitudinal rotation shaft member gradually approach a parallel state as the turn shaft member rotates. This leads to a danger in that foot motions of the user cannot be properly followed, causing the user to feel discomfort.

Moreover, if the lateral rotation shaft member and the longitudinal rotation shaft member reach a parallel state as the turn shaft member rotates, then the lateral rotation shaft member, which is in the topmost position, will become a fourth joint, which cannot be controlled by the drive source. This may cause the turn shaft member, which disposed between the lateral rotation shaft member and the longitudinal rotation shaft member, to easily wobble and become unstable, making it impossible to generate a proper load support force.

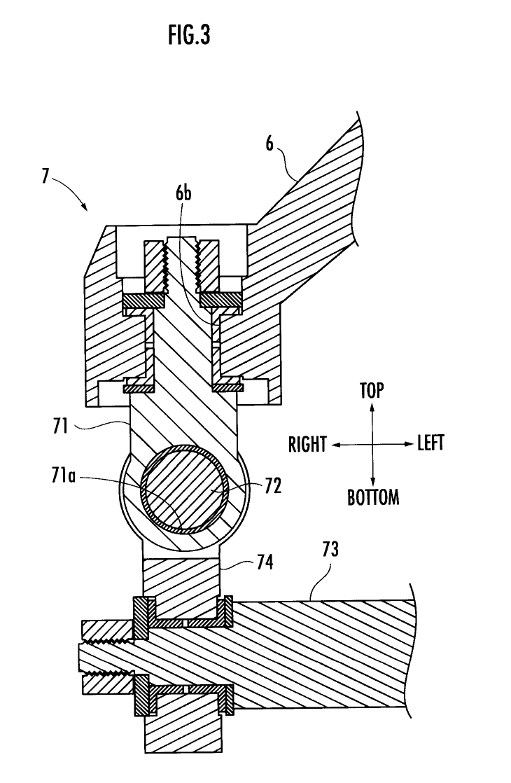

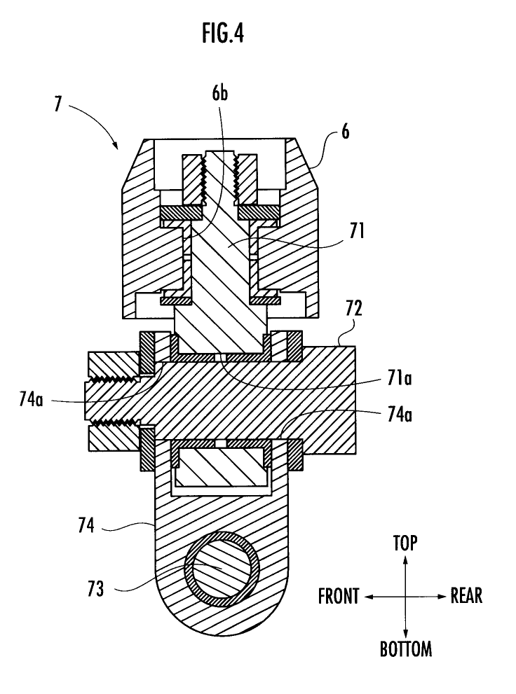

To avoid the aforesaid problem, in the ankle joint 7 of the walking assistance device according to the first embodiment, a turn shaft member 71, a lateral rotation shaft member 72, and a longitudinal rotation shaft member 73 are disposed in this order beginning at the top, as illustrated in FIG. 3 and FIG. 4. Although FIG. 3 illustrates only the ankle joint 7 on the right side, the ankle joint 7 on the left side is constituted in the same manner, the right and left ankle joints 7 and 7 being laterally symmetrical. The right and left ankle joints 7 and 7 would be illustrated identically, so that FIG. 4 illustrates only one of them.

The upper end of the turn shaft member 71 is rotatably supported in a through hole 6b which is drilled at the lower end of the second link member and which vertically penetrates. A lower end portion of the turn shaft member 71 is provided with a through hole 71a, which penetrates in the longitudinal direction, the lateral rotation shaft member 72 being rotationally inserted in the through hole 71a. The lateral rotation shaft member 72 and the longitudinal rotation shaft member 73 are connected by a connection member 74. The upper end portion of the connection member 74 is bifurcate, and the bifurcate upper end portion longitudinally holds the lower end portion of the tarn shaft member 71. The bifurcate portion of the connection member 74 is provided with a through hole 74a, which longitudinally penetrates to match the through hole 71a of the turn shaft member 71, the lateral rotation shaft member 72 being rotatably supported in the through hole 74a. The lower end portion of the connection member 74 is provided with a through hole 74b penetrating in the lateral direction, and the longitudinal rotation shaft member 73 being rotatably supported in the through hole 74b.

With the arrangement described above, the ankle joint 7 maintains a state, wherein the lateral rotation shaft member 72 and the longitudinal rotation shaft member 73 are always orthogonal to each other, regardless of the rotation of the turn shaft member 71, allowing the foot motions of the user to be properly followed. This makes it possible to protect the user from feeling discomfort. Further, the lateral rotation axial line of the lateral rotation shaft member 72 and the longitudinal rotation axial line of the longitudinal rotation shaft member 73 will not become parallel, thus making it possible to obviate the state wherein the fourth joint that cannot be controlled by the drive source develops, as in the case where the turn shaft member is disposed between the lateral rotation shaft member and the longitudinal rotation shaft member. Hence, an appropriate load support force can be always generated.

The turn shaft member 71 may alternatively be disposed at the bottommost position and connected to the ground contact member 8. Further, the lateral rotation shaft member 72 and the longitudinal rotation shaft member 73 may be vertically switched and the lateral rotation shaft member 72 may be connected to the ground contact member 8.

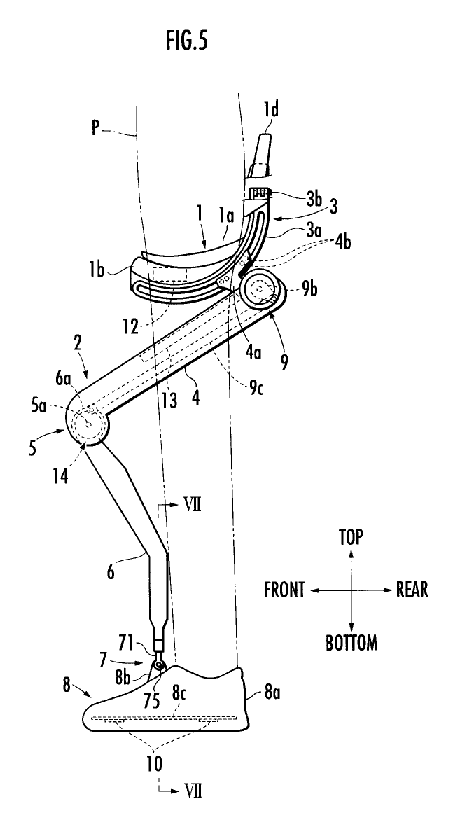

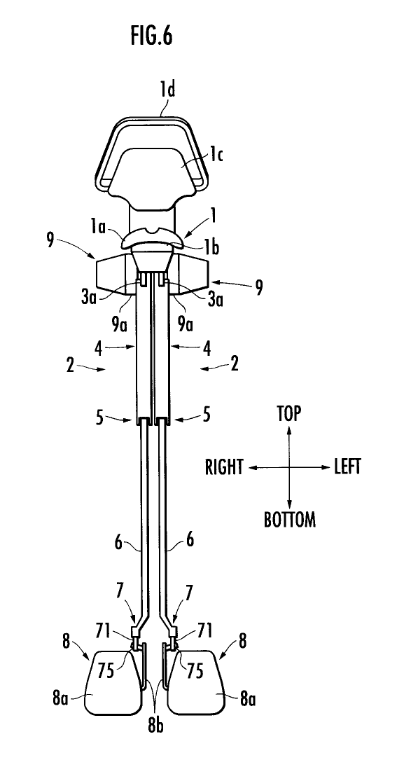

Referring now to FIG. 5 through FIG. 7, a walking assistance device to which a second embodiment of the ankle joint structure in accordance with the present invention has been applied will be described. As illustrated in FIG. 5 and FIG. 6, the walking assistance device according to the second embodiment is exactly the same as the first embodiment except for the construction of an ankle joint 7.

The ankle joint 7 of the second embodiment has a spherical joint 75. Here, the spherical joint in its discrete form has three degrees of freedom. This is advantageous for achieving compactness, while presenting a problem in that operating angles are small, making it impossible to properly follow the foot motions of the user.

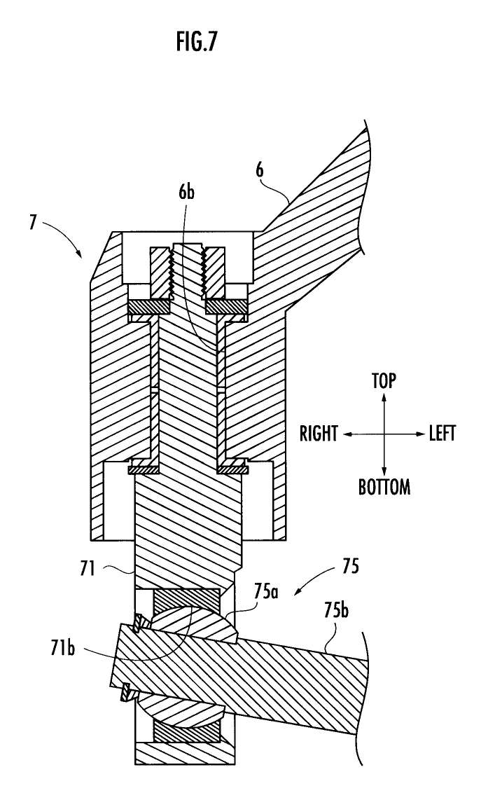

Hence, as illustrated in FIG. 7, the ankle joint 7 of the second embodiment is provided with a turn shaft member 71 having its upper end turnably connected to a leg link 2, in addition to the spherical joint 75. A retaining hole 71b laterally penetrating is formed in the lower end portion of the turn shaft member 71, the retaining hole 71b swingably retains a sphere 75a of the spherical joint 75, and a joint shaft member 75b of the spherical joint 75 is connected to the ground contact member 8. Although FIG. 7 illustrates only the ankle joint 7 on the right side, the ankle joint 7 on the left side is constituted in the same manner, the right and left ankle joints 7 being laterally symmetrical.

Thus, the turn shaft member 71 ensures the rotation of a foot in a turning direction, which is larger than a lateral rotation and a longitudinal rotation of a foot of the user, while the spherical joint 75 ensures the lateral rotation and the longitudinal rotation, which are relatively smaller than the rotation of a foot in the turning direction. This arrangement makes it possible to configure the ankle joint 7 capable of following the foot motions of a user so properly and smoothly that the user does not feel uncomfortable.

The joint shaft member 75b of the spherical joint 75 is constructed such that an inner end portion thereof connected to the ground contact member 8 is positioned below an outer end portion of the joint shaft member 75b. Foot motions of a human being in many cases cause the leg link 2 to incline inward from an upright state, as observed from the front, when, for example, the user opens his/her legs apart, and an angle formed by the leg link 2 inclining outward is not so large, as observed from the front. Hence, in the upright posture state, tilting the joint shaft member 75b such that the inner end portion thereof is positioned below the outer end portion thereof enables the leg link 2 to take a large angle (operating angle) when tilting inward relative to the ground contact member 8, thus making it easy to further properly follow the foot motions of the user.

Alternatively, the turn shaft member 71 may be connected to the ground contact member 8 and the spherical joint 75 may be connected to the leg link 2. In this case, the joint shaft member 75b of the spherical joint 75 may be constructed such that the inner end portion thereof is positioned above the outer end portion thereof. This enables the user to open his/her legs wide apart, making it possible to further properly follow the foot motions of the user.

What is claimed is:

1. An ankle joint structure of a walking assistance device which is used with a walking assistance device having a load transmit assembly, a ground contact assembly, and a leg link provided between the load transmit assembly and the ground contact assembly to support at least a part of the weight of a user by the leg link through the intermediary of the load transmit assembly, and which connects the ground contact assembly and the leg link, the ankle joint structure comprising:
a turn shaft member that enables the ground contact assembly to turn relative to the leg link;
a longitudinal rotation shaft member that enables the ground contact assembly to rotate in a longitudinal direction relative to the leg link; and
a lateral rotation shaft member that enables the ground contact assembly to rotate in a lateral direction relative to the leg link,
wherein the turn shaft member is disposed above or below the longitudinal rotation shaft member and the lateral rotation shaft member.

2. An ankle joint structure of a walking assistance device which is used with a walking assistance device having a load transmit assembly, a ground contact assembly, and a leg link provided between the load transmit assembly and the ground contact assembly to support at least a part of the weight of a user by the leg link through the intermediary of the load transmit assembly, and which connects the ground contact assembly and the leg link through toe intermediary of a spherical joint, the ankle joint structure comprising:
a turn shaft member that enables the ground contact assembly to turn relative to the leg link,
wherein one end of the turn shaft member is connected to one of the ground contact assembly and the leg link, while the other end of the turn shaft member swingably retains a sphere of the spherical joint, and a joint shaft member provided on the sphere is connected to the other of the ground contact assembly and the leg link.
